(19) United States
(12) Patent Application Publication
(10) Pub. No.: US 2025/0029711 A1
(43) Pub. Date: Jan. 23, 2025

(54) METHOD AND SYSTEM FOR AUTOMATED RELATIONSHIP DISCOVERY IN A PHARMACEUTICAL NETWORK

(71) Applicant: Express Scripts Strategic Development, Inc., St. Louis, MO (US)

(72) Inventors: Paul T. Anderson, Chanhassen, MN (US); Zachary P. Curtin, Feeding Hills, MA (US)

(21) Appl. No.: 18/223,837

(22) Filed: Jul. 19, 2023

(51) Int. Cl. G16H 40/20; G16H 50/20

(52) U.S. Cl. CPC G16H 40/20; G16H 50/20

(57) ABSTRACT

A system and method for automated relationship discovery in a pharmaceutical network may include analyzing human-readable text in messages associated with computing devices in a pharmaceutical network. The system and method may include determining a relationship between a first computing device of the computing devices and one of (i) a first computer program operated by the first computing device or (ii) a second computing device of the computing devices, based on the analysis of the human-readable text, and updating a configuration management database (CMDB) to include a representation of the relationship that is determined.

### BACKGROUND

A pharmaceutical managed network may include thousands of individual computing devices to efficiently facilitate and manage many interrelated pharmaceutical operations, including processing and filling a large number of prescriptions and prescription orders. For example, some computing devices may be associated with pharmacies, other computing devices may be associated with a benefit manager, and still other computing devices may be associated with a fulfillment center. Each computing device operates based on programmed instructions (e.g., software), referred to herein as computer programs. To track the topology of the pharmaceutical managed network, pharmaceutical managed networks may include configuration management databases (CMDB). A CMDB is a repository of information about the relationships between the physical/logical machines in the managed network and the application service items that run on the machines. For example, a CMDB may include representations of the computing devices in the managed network, the computer programs operated by the computing devices, relationships among the computing devices, relationships between the computing devices and the computer programs, and configurations thereof. The information stored in the CMDB can be utilized to perform various operations on the managed network, such as information technology (IT) service management (e.g., network monitoring), operations management, asset management, configuration management, compliance, and the like. Inaccuracies and information gaps in the CMDB may negatively affect the efficiency with which these operations are performed.

CMDB repositories and the component relationships contained in the CMDB repositories can be difficult to maintain due at least to the size of some managed networks and frequency of changes in the managed networks. Traditionally, an operator may manually update a CMDB by accessing the CMDB and inputting information to describe the components of the managed network and relationships between the components. As such, the CMDB is only periodically updated when the operator takes the time to manually input information. Updating the CMDB can be difficult as well due to complex interrelations between components. For example, if a new computing device is added to the managed network to replace an old computing device, there may be substantial re-work to be performed in the CMDB to establish new relationships and data and remove outdated relationships and data.

Furthermore, it may be difficult to ascertain information needed to build and complete the network topology in the CMDB. For example, manual set-up of a new computing device in the managed network may involve a human operator hand-coding information corresponding to relationships among the new computing device, other computing devices in the network, and computer programs operated by the new computing device. The hand-coded relationships input during the set-up process may not be entered into the CMDB, and this information may be lost over time as devices are updated and replaced. As a result, the information contained in the CMDB may be outdated, incomplete, and/or incorrect due to human error. A need may exist for a system and method that automatically discovers relationships between components to update and validate the information contained in the CMDB.

### BRIEF DESCRIPTION

In one embodiment, a method for automated discovery of component relationships in a pharmaceutical network includes obtaining messages associated with computing devices in a pharmaceutical network; analyzing, via a monitoring device comprising one or more processors, human-readable text in the messages; determining, via the monitoring device, a relationship between a first computing device of the computing devices and one of (i) a first computer program operated by the first computing device or (ii) a second computing device of the computing devices, based on the analysis of the human-readable text; and updating a configuration management database (CMDB) to include a representation of the relationship that is determined.

In another embodiment, a network management system for a pharmaceutical network includes a configuration management database (CMDB) that contains information related to computing devices in a pharmaceutical network, computer programs operated by the computing devices, and relationships among the computing devices and the computer programs; and a monitoring device comprising one or more processors and communicatively connected to the CMDB, the monitoring device configured to obtain messages associated with the computing devices in the pharmaceutical network and analyze human-readable text in the messages, the monitoring device configured to determine a relationship between a first computing device of the computing devices and one of (i) a first computer program operated by the first computing device or (ii) a second computing device of the computing devices, based on the analysis of the human-readable text, the monitoring device configured to update the CMDB to include a representation of the relationship that is determined.

In a further embodiment, a computer program product includes a non-transitory computer readable storage medium, the non-transitory computer readable storage medium comprising computer executable code configured to be executed by one or more processors to: obtain messages associated with computing devices in a pharmaceutical network; analyze human-readable text in the messages; determine a relationship between a first computing device of the computing devices and one of (i) a first computer program operated by the first computing device or (ii) a second computing device of the computing devices, based on the analysis of the human-readable text; and update a configuration management database (CMDB) to include a representation of the relationship that is determined.

### BRIEF DESCRIPTION OF THE DRAWINGS

FIG. 1 illustrates a managed pharmaceutical network according to an embodiment.

FIG. 2 is a block diagram of a portion of a pre-existing CMDB at a first time prior to the monitoring device performing the automated discovery process according to an embodiment.

FIG. 3 is a block diagram of a portion of an updated CMDB at a second time after the monitoring device performs the automated discovery process according to an embodiment.

FIG. 4 illustrates a flowchart of one example of a method for automated relationship discovery in a pharmaceutical network.

FIG. 5 shows a block diagram of a monitoring device in the example form of a computer system within which a set of instructions may be executed causing the machine to perform any one or more than one methods, processes, operations, or methodologies discussed herein.

FIG. 6 illustrates a functional block diagram of an example neural network that can be used by the monitoring device for analyzing human-readable text within messages to determine relationships among the computing devices and computer programs in the pharmaceutical network.

In the drawings, reference numbers may be reused to identify similar and/or identical elements.

### DETAILED DESCRIPTION

The subject matter described herein provides a network management system and method that can discover relationships between components in a pharmaceutical network. The network management system and method can use the relationships that are discovered to update a database that describes a topology of the pharmaceutical network. The database is referred to herein as a CMDB. The network management system and method may automatically discover the relationships using low level information including messages associated with one or more of the computing devices in the pharmaceutical network. For example, information that describes or relates to the rudimentary, daily operations of individual computing devices in the network may be low level information. The relationships may be discovered by analyzing human-readable text contained in the messages. Human-readable text refers to information that can be naturally read by humans without requiring translation, decoding, or other transformation in order to interpret the text. For example, strings of alphanumeric characters that form words and phrases may be human-readable text. Human-readable text may be text that is in a spoken language. The human-readable text may follow the syntax of the spoken language. In an example, binary and hexadecimal language may not constitute human-readable text.

The messages may be unrelated to database management, yet the network management system and method may extract contextual information from the messages used to automatically update and/or validate information stored in the CMDB. For example, the network management system and method may determine, via the analysis of the human-readable text in the messages, a first relationship between a first computing device and a second computing device in the pharmaceutical network. If there is no pre-existing relationship between the first and second computing devices in the CMDB, the network management system and method may create a representation of the first relationship within the CMDB. If, on the other hand, the CMDB already includes a stored representation of a relationship between the first and second computing devices, the network management system and method may compare the first relationship that is determined to the pre-existing relationship for validation.

Databases that track the devices, programs, and component relationships of large networks are difficult to maintain over time. Furthermore, such databases are typically manually updated which is inefficient, untimely, and error prone. The network management system and method described herein remedies these shortcomings by automatically augmenting and/or validating the network topology information stored in the CMDB using the content of available messages. More specifically, the network management system and method extracts information about the relationships between different components of the network from human-readable text within messages associated with one or more of the computing devices. The network management system and method may not be constrained by the limited availability, inefficiency, and errors inherently attributable to manual data entry. Furthermore, a monitoring device of the network management system and method may be able to decipher component relationships by analyzing the messages, which a human operator may not be able to decipher. For example, due to the greater processing speed and memory storage capacity of the monitoring device over the human operator, the monitoring device may be able to recognize connections or relationships between components that the human operator may fail to ascertain, or may only ascertain after a much longer delay relative to the monitoring device.

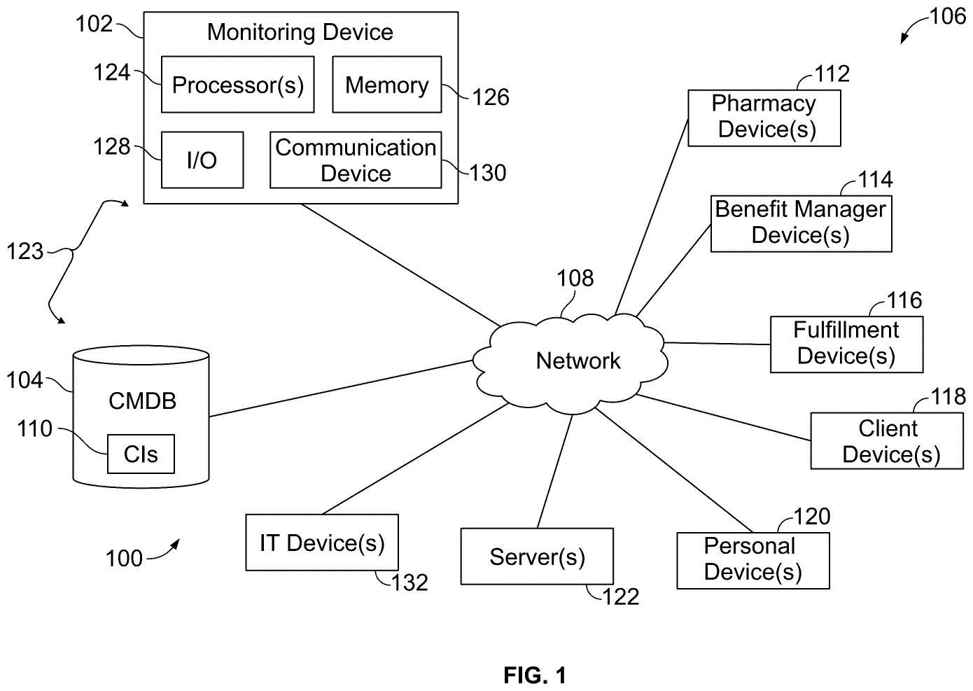

FIG. 1 illustrates a managed pharmaceutical network 100 according to an embodiment. The pharmaceutical network 100 includes a monitoring device 102, a CMDB 104, and a plurality of computing devices 106. The computing devices 106, monitoring device 102, and CMDB 104 can be communicatively connected via one or more communication networks 108. In an embodiment, the monitoring device 102 may obtain and analyze messages associated with one or more of the computing devices 106 in the pharmaceutical network 100 to discern relationships related to the computing devices 106, the computer programs operated by the computing devices 106, and/or the like. The monitoring device 102 may use the relationships that are discerned based on the messages to update and/or validate the CMDB 104.

The CMDB 104 of the embodiments described herein may be a repository of information about the relationships between the computing devices 106 in the pharmaceutical network 100 and the application service items that run on the computing devices 106. Although the CMDB 104 is shown in FIG. 1 as a standalone component, the CMDB 104 may be a sub-component in a storage device. For example, the CMDB 104 may be a component of a server or computer device (such as a portion of a memory or hard drive). Optionally, the CMDB 104 may represent the server or computing device itself. The CMDB 104 may include representations of the computing devices 106 in the pharmaceutical network 100, the computer programs operated by the computing devices 106, relationships among the computing devices 106, relationships between the computing devices 106 and the computer programs, configurations thereof, or the like.

The information stored in the CMDB 104 can be utilized to perform various operations on the pharmaceutical network 100, such as information technology (IT) service management (e.g., network monitoring), operations management, asset management, configuration management, compliance, and the like. The monitoring device 102 may perform an automated discovery operation to update and/or validate the information stored in the CMDB 104. The monitoring device 102 ensures that the CMDB 104 is accurate and up-to-date, which benefits the operations that rely on the CMDB 104. For example, an IT service management application may use information in the CMDB 104 to determine applications and services that may be impacted by a component (e.g., a server device) that has malfunctioned, crashed, or is heavily loaded. Likewise, an asset management application may use information in the CMDB 104 to determine which hardware and/or software components are being used to support particular enterprise applications. As a consequence of the importance of the CMDB 104, it is desirable for the information stored therein to be accurate, consistent, and up-to-date.

The computer programs are program instructions (e.g., software) that are implemented by processing circuitry of the computing devices 106. The computer programs may include applications, operating systems, services, and/or the like. In an example, at least some of the computer programs may be locally stored in the individual computing devices 106. An application may refer to software that executes on a device or group of devices. A service may refer to a high-level capability provided by multiple applications executing on one or more computing devices working in conjunction with one another. For example, a high-level web service may involve multiple web application server threads executing on one device and accessing information from a database application that executes on another device.

The CMDB 104 stores representations of the computing devices 106, computer programs, and relationships to provide topological information. Topological information includes an identification of the components of the pharmaceutical network (e.g., the computing devices 106, the computer programs operated by the computing devices 106, etc.) and known relationships between the components. The topological information may be presented as a map or map-like diagram on a graphical user interface. The representations of the computing devices 106 and the computer programs in the CMDB 104 may be referred to as configuration items 110 (e.g., “CIs” in FIG. 1). For example, each computing device 106 in the pharmaceutical network 100 may constitute a discrete configuration item 110 that is stored in the CMDB 104. A first configuration item 110 may be a first computing device 106, and a second configuration item 110 may be the operating system that is run on the first computing device 106. A third configuration item 110 in the CMDB 104 may be an application that is operated by the first computing device 106 to communicate with other computing devices 106 in the pharmaceutical network 100. The term “configuration items” may refer to physical or virtual devices (e.g., client devices, server devices, routers, or virtual machines) and/or computer programs executing thereon (e.g., web servers, email servers, etc.). The CMDB 104 may represent a repository of configuration items 110 and the relationships between the configuration items 110.

The relationships between the configuration items 110 (e.g., the components of the pharmaceutical network 100) in the CMDB 104 may be entries that describe associations between configuration items 110. The associations are indicative of interrelations within the pharmaceutical network 100. The associations may be dependencies between computing devices 106. The dependencies may include manager-follower relationships. For example, the relationships may indicate that a first group of computing devices 106 is controlled or managed by a particular host computing device 106. If the host computing device 106 is taken out of operation for maintenance, the first group of computing devices 106 may all be impacted. Furthermore, a relationship entry in the CMDB 104 may indicate an application that is executing on a particular computing device 106, as well as the services that rely on this application. If a database application executing on a server device is used for a prescription order service, if the server device is taken out of operation for maintenance, it is clear from the stored relationship in the CMDB 104 that the prescription order service would be impacted. Optionally, the relationships between configuration items 110 in the CMDB 104 may be able to indicate the services impacted when a particular router fails. The configuration items 110 and relationships in the CMDB 104 may form a topological map (or web) representative of the pharmaceutical network 100. The monitoring device 102 may perform the discovery process described herein to repeatedly update and/or validate the topological map of the pharmaceutical network 100 provided by the CMDB 104.

The computing devices 106 of the pharmaceutical network 100 illustrated in FIG. 1 include one or more pharmacy devices 112, one or more benefit manager devices 114, one or more fulfillment devices 116, one or more client devices 118, one or more personal devices 120, one or more servers 122, and one or more IT support devices 132. Each pharmacy device 112 may be associated with a retail pharmacy location (e.g., an exclusive pharmacy location, a grocery store with a retail pharmacy, or a general sales store with a retail pharmacy) or other type of pharmacy location at which a member attempts to obtain a prescription. The pharmacy may use the pharmacy device 112 to submit the claim to a pharmacy benefit manager for adjudication. The pharmacy device 112 may be used by a pharmacist and/or pharmacy technician. The pharmaceutical network 100 may include a plurality of different pharmacies, and each pharmacy may include one or more pharmacy devices 112.

Each benefit manager device 114 is a device operated by an entity that is at least partially responsible for creation and/or management of the pharmacy or drug benefit. The entity operating the benefit manager device 114 is typically a pharmacy benefit manager (PBM). Optionally, other entities may operate the benefit manager device 114 on behalf of themselves or other entities (such as PBMs). For example, the benefit manager device 114 may be operated by a health plan, a drug wholesaler, a data analytics or other type of software-related company, and/or the like.

Each fulfillment device 116 may be used to process and fulfill prescriptions and prescription orders. For example, a fulfillment device 116 may fulfill, dispense, aggregate, and/or pack the order components of the prescription drugs in accordance with one or more prescription orders directed by the pharmacy device 112. Optionally, a fulfillment device 116 may include one or more sub-components, such as a loading device, an inspection device, an automated dispensing device, an imaging device, a cap device, an accumulation device, a packing device, a literature device, a mail manifest device, and/or the like.

Each client device 118 may be used by a client of a pharmacy, such as a patient. The client device 118 may be a desktop computer, a laptop computer, a tablet computer, a smartphone, or the like. The client may use the client device 118 to access information about a prescription, a prescribed medication, billing related to a prescribed medication, insurance, or the like. Each personal device 120 may be used by an operator affiliated with the pharmaceutical network 100, such as a pharmacist, a data analyst, a health plan administrator, or the like. The personal device 120 may be a personal computing device such as a smartphone, laptop computer, tablet computer, desktop computer, or the like. An operator may use a corresponding personal device 120 to access the benefit manager device 114, the pharmacy device 112, and/or the fulfillment device 116 from a remote location. The pharmaceutical network 100 optionally may include one or more servers 122 or other computer-readable storage devices. The one or more servers 122 may store order data, client data, prescription data, claims data, drug data, plan sponsor data, and/or the like. Each IT support device 132 may be used by an IT professional that is affiliated with the pharmaceutical network 100. For example, the IT support devices 132 may be used to access other computing devices 106 in the pharmaceutical network 100 for maintenance and repair, as well as to set up new computing devices 106 in the pharmaceutical network 100 and remove selected existing computing devices 106 from the pharmaceutical network 100. The IT support devices 132 may be part of an in-house IT department or an external IT company.

The communication network(s) 108 may be a local area network or a global communication network, such as the Internet. In an example, the communication network(s) 108 may include a Global System for Mobile Communications (GSM) network, a code division multiple access (CDMA) network, a 3rd Generation Partnership Project (3GPP), an Internet Protocol (IP) network, an optical network, a Wireless Application Protocol (WAP) network, an IEEE 802.11 standards network, and/or the like, including a combination thereof. Although FIG. 1 shows a single network 108, multiple networks 108 can be used. The multiple networks 108 may communicate with each other to link the devices 102, 104, 106. The devices 102, 104, 106 may be communicatively connected to one another through the network 108 over wired and/or wireless communication pathways (e.g., links).

The monitoring device 102 may be a computing device 106 that performs at least some of the automated relationship discovery operations for updating the CMDB 104 described herein. For example, the monitoring device 102 and the CMDB 104 may represent a network management system 123 for the pharmaceutical network 100. The monitoring device 102 and the CMDB 104 function to manage the operations of the pharmaceutical network 100 and the computing devices 106 within the pharmaceutical network 100.

The monitoring device 102 may include one or more processors 124, a memory device 126, an input/output device 128, and a communication device 130. The one or more processors 124 may represent a controller (e.g., control circuit) of the monitoring device 102. For example, the controller may represent hardware circuitry that includes and/or is connected with a single processor or multiple processors (e.g., one or more microcontrollers, integrated circuits, field programmable gate arrays, microprocessors, etc.) that perform the operations described in connection with the monitoring device 102. As described herein, the processor(s) of a device may each perform all of the functions or operations of the device, or different processors may perform at least one different function or operation of the device relative to at least one other processor of the same device. Stated differently, multiple processors may perform different operations or may perform some of the same operations. The monitoring device 102 may be communicatively connected to the CMDB 104 via a direct communication pathway or indirectly via the communication network(s) 108. In an example, the monitoring device 102 may be integrated onto a server that stores and/or monitors messages communicated in the pharmaceutical network 100. In another example, the monitoring device 102 may be a discrete computing device 106 that communicates with a server 122 to obtain the messages communicated in the pharmaceutical network 100. The memory device 126 may include a tangible and non-transitory computer-readable storage medium. The memory device may store programmed instructions (e.g., software) that are executed by the one or more processors to perform the discovery operations described herein. The programmed instructions may include one or more natural language processing algorithms and/or machine learning algorithms utilized by the one or more processors.

The input/output device 128 may be used to receive information from an operator (e.g., a human user) and/or provide information to the operator. In some examples, input/output device may include a display device, an audio output device, a light arrangement, or the like. The display device may be a liquid crystal display (LCD), organic light emitting diode (OLED) display, “electronic ink” display, or the like. The audio output device may be a speaker or headphones. The light arrangement may be a light-up dashboard at a control center. Optionally, the output device may generate a graphical user interface (GUI) that is presented on the display device for viewing by an operator. The GUI may present a representation or map of the configuration items 110 and relationships between the configuration items 110. The input/output device may include one or more devices for receiving input from the operator. Such input devices may include, for example, a keyboard, a mouse, a stylus, a button, a joystick, a touch screen, a touch pad, or an audio input device. A single component, such as a touch screen, may function as both an output device and input device.

The communication device 130 may represent hardware circuitry that can communicate signals via wired and/or wireless communication pathways. The communication device may include a wired or wireless network adapter or a wireless data transceiver for use with a mobile phone network, Global System for Mobile communications (GSM), 3G, 4G, or other mobile data network or Worldwide Interoperability for Microwave Access (WIMAX). The communication device may allow the monitoring device 102 to interface with other computing devices 106 in the pharmaceutical network 100 using an appropriate wireless or wired communications protocol such as, without limitation, Bluetooth®, Ethernet, or IEE 802.11. Optionally, the communication device may include transceiving circuitry (e.g., a transceiver or a receiver and discrete transmitter), one or more antennas, and the like for radio communication.

The monitoring device 102 may perform an automated relationship discovery process to determine relationships among the computing devise and the computer programs in the pharmaceutical network 100. The monitoring device 102 may perform the discovery process periodically at a designated interval. For example, the monitoring device 102 may perform the discovery process once per hour, once a day, once a week, or the like. Optionally, the monitoring device 102 may perform the discovery process on-demand in response to receiving a discovery request command from the input/output device 128 or the communication device 130.

The discovery process involves the monitoring device 102 obtaining messages associated with the computing devices 106 in the pharmaceutical network 100. The monitoring device 102 may obtain and analyze various different types of messages. At least some of the messages may be communications between two or more computing devices 106 in the pharmaceutical network 100. A first message may be generated and transmitted from the first computing device 106 to a second computing device 106. A second message may be generated and transmitted from the second computing device 106 to a third computing device 106. The first and second messages that are communicated may be SMS text messages, Bluetooth messages, email messages, or messages transmitted in an application-specific format. For example, a pharmacist may use one of the pharmacy devices 112 to access a specific pharmacy application. The pharmacist may generate the first message using the pharmacy application. The first message may be transmitted in a format specific to the pharmacy application. In an example, the first message is transmitted to the benefit manager device 114, which represents the second computing device 106. The first message may be an order related to a prescription (e.g., a prescription-related order). In an example, the second message may be an order to fill the prescription that is sent from the benefit manager device 114 to a fulfillment device 116 after the benefit manager device 114 verifies the prescription.

In an example, the messages obtained by the monitoring device 102 for the discovery process may not be limited to bi-directional communications between two communicating devices. For example, the messages may include notes that are generated by a pharmacist, an IT engineer, a client, a benefit manager, and/or the like. The notes may only be communicated from the source computing device 106 that generates the notes to one or more of the remote servers 122 or other storage devices for record-keeping. The notes may include observations, details, thoughts, and/or the like of the human operator that generates the notes.

The monitoring device 102 may obtain the messages by accessing one or more remote servers 122 or other storage devices that maintain a record of activity in the pharmaceutical network 100. The monitoring device 102 may remotely access the remote servers 122 or other storage devices via the communication network(s) 108. Optionally, the monitoring device 102 may obtain the messages by accessing local memory devices on individual computing devices 106 in the pharmaceutical network 100. In addition, the monitoring device 102 may be able to intercept communications that are transmitted through the pharmaceutical network 100 in real time. For example, the monitoring device 102 may intercept a message by receiving a copy of the message, analyzing all traffic received by one or more specific nodes (e.g., routers) in the communication network 108, and/or the like. In an example, the monitoring device 102 may obtain email messages that are associated with a user of a first computing device 106. The monitoring device 102 may obtain the email messages by accessing a stored record of the email messages via an email account that is associated with the user and/or intercepting the email messages during transmission. In another example, the monitoring device 102 may obtain messages that are generated by a specific application operated by one or more of the computing devices 106. The monitoring device 102 may obtain these application-specific messages by accessing a remote database or server 122 that stores a record of the application-specific messages generated by the one or more computing devices 106 over time.

Example types of messages obtained by the monitoring device 102 may include prescription-related orders, IT service requests, engineer notes, pharmacist notes, and the like. The prescription-related orders broadly encompasses messages related to prescribing medication, dispensing the medication, verifying prescriptions are covered in a health plan or insurance plan, and/or the like. The IT service requests refer to messages requesting IT assistance, such as to address an IT issue, set up a new computing device 106 in the pharmaceutical network 100, set up a new computer program on one of the computing devices 106, and/or remove an existing computing device 106 from the pharmaceutical network 100. The engineer notes may refer to notes generated by an IT engineer during a set-up procedure. For example, during initial set-up of a new computing device 106 on the pharmaceutical network 100, the IT engineer may make note of several details. The details may include several associations that can be used to establish relationships in the CMDB 104. For example, the details provided in engineer notes may include the identity and type of the new computing device 106, the type of computer programs operated by the new computing device 106, and/or an association between the new computing device 106 and one or more other computing devices 106. In an example, the engineer may make a note that the new computing device 106 is in a first group of pharmacy devices 112, which all have a child relationship to a host pharmacy device 112.

The messages obtained by the monitoring device 102 include human-readable text, which is information that can be naturally read by humans. For example, the human-readable text includes character strings that form words and phrases. The character strings include alphanumeric characters. The human-readable text may be located within one or more component parts of the respective message. For example, a first message may include a sender field, a recipient field, a subject field, a body field, and/or a signature field. The sender field may describe the source device, account, and/or user that generates the first message. The recipient field may describe a target device, account, and/or user that is intended to receive the first message. The subject field may describe an object, theme, or purpose of the first message. The body field may provide the primary descriptive contents of the first message. The signature field may identify a user that formulated the body of the first messages. The monitoring device 102 may scan the various component parts of the messages to detect human-readable text. For example, the monitoring device 102 may scan the various fields of the first message to detect alphanumeric characters strings indicative of human-readable text. After detecting human-readable text in one or more of the fields, the monitoring device 102 may analyze the human-readable text to interpret the human-readable text.

In an example, the monitoring device 102 may also inspect metadata in the messages for the presence of human-readable text. For example, one or more of the messages may include human-readable metadata, such as XML data. The monitoring device 102 may analyze the human-readable text in the metadata as well as the human-readable text in other fields or parts of the messages.

The monitoring device 102 may analyze the human-readable text in the messages by implementing one or more natural language processing techniques (e.g., algorithms) and/or providing the human-readable text as an input to a machine learning algorithm. In a first embodiment, the monitoring device 102 may implement one or more natural language processing techniques without using a machine learning algorithm. The one or more natural language processing techniques may be performed using a natural language engine incorporated into the monitoring device 102. In a second embodiment, the monitoring device 102 may provide the human-readable text as an input to a machine learning algorithm, without using a natural language processing technique. The machine learning algorithm may be an artificial neural network or the like. The machine learning algorithm may be incorporated into the monitoring device 102 or, alternatively, may be remote from the monitoring device 102. In a third embodiment, the monitoring device 102 may utilize both the machine learning algorithm and the one or more natural language processing techniques to analyze the human-readable text.

Based on the analysis of the human-readable text, the monitoring device 102 may determine one or more relationships between computing devices 106 and/or computer programs. For example, the monitoring device 102 may determine a first relationship between a first computing device 106 in the pharmaceutical network 100 and a second computing device 106 in the pharmaceutical network 100. The first relationship may indicate or describe how the first computing device 106 is associated with the second computing device 106. For example, the first and second computing devices 106 may be in a manager-follower relationship, such that the first computing device 106 controls and/or manages operations of the second computing device 106 and/or the second computing device 106 reports to the first computing device 106. In another example, the first relationship may indicate that the first computing device 106 is a benefit manager device 114 that is associated with a pharmacy device 112 that represents the second computing device 106. In another example, the monitoring device 102 may determine a second relationship between the first computing device 106 and a computer program operated by the first computing device 106, based on the analysis of the human-readable text. The second relationship in this example indicates that the first computing device 106 includes and/or is operating the computer program. Therefore, once the CMDB 104 is updated to include a representation of the second relationship, an operator can access the CMDB 104 to determine which computing devices 106 include that computer program for performing a software update or the like.

The monitoring device 102 may determine the relationships (e.g., the first and second relationships in the examples above), by ascertaining meaning and context from the human-readable text in one or more of the messages. In a simple example, first human-readable of a first message may explicitly state that a first computing device 106 is used to generate the first message, and second human-readable text in the first message may state which application program (e.g., application, operating system, etc.) is operated by the first computing device 106. The first human-readable text that identifies the first computing device 106 may be within the sender field, the signature field, the metadata, and/or the like. The second human-readable text that identifies the program may be within the metadata, such as an identifier of the application format of the message. In the case of an IT service request, the body of the first message may identify the computer program. For example, the IT service request may state that a certain program is not properly functioning, and may ask for an IT engineer to perform troubleshooting to address the issue. The monitoring device 102 may be programmed and/or trained (e.g., in the case of a machine learning algorithm) to ascertain relationships based on certain types of information gleaned from the human-readable text.

An example use case for the network management system may be to add a pharmacist computer to the pharmaceutical network 100. The new computer will operate an application connected to a server 122 of the pharmaceutical network 100. The monitoring device 102 may be incorporated into the server 122, or may be discrete from the server 122 and communicatively connected to the server 122. The monitoring device 102 may obtain messages that are communicated to and from the new pharmacist computer via the application. A first message may be the pharmacist communicating with a benefit manager device 114. A second message may be the pharmacist communicating with an IT support device 132. A third message may include notes that were generated by an engineer while setting up the new pharmacist computer. The monitoring device 102 may inspect the first, second, and third messages for the presence of human-readable text, and then analyze that human-readable text to determine one or more relationships among the new pharmacist computer, the application operated by the new pharmacist computer, the benefit manager device 114, and/or the IT support device 132.

After determining one or more relationships based on the human-readable text, the monitoring device 102 may update the CMDB 104 to include a representation of each of the one or more relationships that are determined. The monitoring device 102 may update the CMDB 104 by creating (e.g., generating) a new association between two configuration items 110 in the CMDB 104. Optionally, the monitoring device 102 may update the CMDB 104 by validating an existing association between two configuration items 110. For example, after determining a relationship between a first computing device 106 and a second computing device 106, the monitoring device 102 may access the respective representations of the first and second computing devices 106 in the CMDB 104. If the CMDB 104 lacks any existing relationship between the first and second computing devices 106, then the monitoring device 102 may automatically generate a new relationship representation that is stored in the CMDB 104. On the other hand, if the CMDB 104 already contains a representation of the relationship between the first and second computing devices 106, the monitoring device 102 may compare the newly-determined relationship, which is based on the human-readable text, to the pre-existing relationship. If the two relationships match, then the monitoring device 102 may add a note to the CMDB 104 that the relationship between the first and second computing devices 106 is valid as of the current date. If, on the other hand, the two relationships differ, then the monitoring device 102 may update the CMDB 104 to add a representation of the newly-determined relationship in place of the representation of the pre-existing relationship. The pre-existing relationship may be incorrect due to human operator error, a change in the pharmaceutical network 100 that has not been incorporated into the CMDB 104, or the like. The monitoring device 102 may add new relationships and fix incorrect relationships in the CMDB 104.

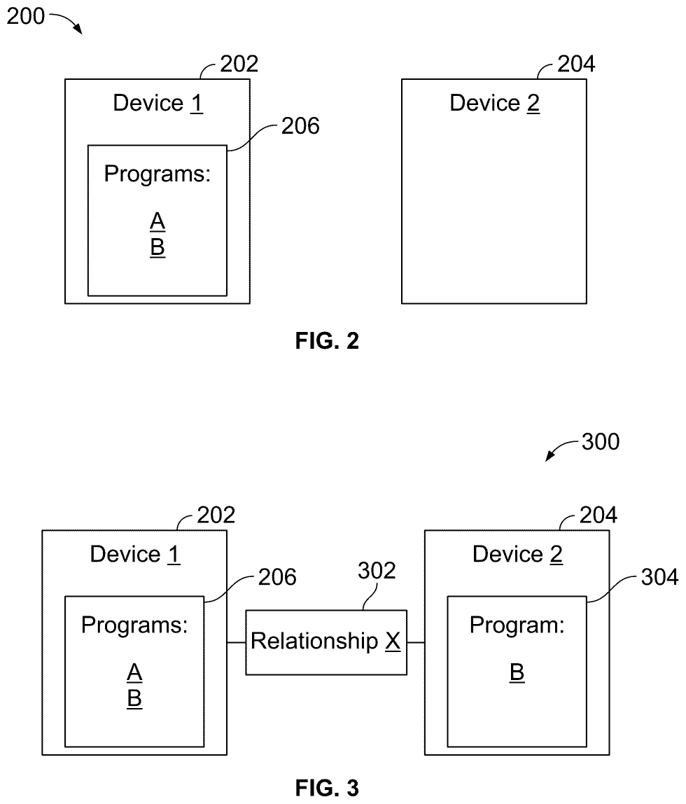

FIG. 2 is a block diagram of a portion of a pre-existing CMDB 200 at a first time prior to the monitoring device 102 performing the automated discovery process according to an embodiment. The CMDB 200 includes a representation 202 of a first computing device 106 (e.g., “Device 1”) in the pharmaceutical network 100 and a representation 204 of a second computing device 106 (e.g., “Device 2”) in the pharmaceutical network 100. The CMDB 200 also includes a representation 206 of two computer programs (e.g., Programs A and B) operated by the first computing device 106. The first computing device 106 indicated by the representation 202, the second computing device 106 indicated by the representation 204, and the programs indicated by the representation 206 are configuration items 110 (shown in FIG. 1). The representations 202, 204, 206 may be data or information that is stored in the CMDB 200 as a proxy for the corresponding configuration items. The representations in the CMDB 200 are associated with different corresponding configuration items 110. For example, the CMDB 200 may include data or information (e.g., the representation 206) which is indicative of (e.g., refers to) Program A without actually including all of the data or information that constitutes Program A.

Optionally, the CMDB 200 may be displayed on a display device for viewing by an operator. The operator may view the representations 202, 204, 206 (and other representations of the configuration items 110) depicted in a GUI that is displayed on the display device. In the GUI, the representations 202, 204, 206 may be graphic indicia that are labeled or otherwise indicated as being associated with the different corresponding configuration items 110. For example, the representation 202 may be labeled Device 1 to show association with the first computing device 106, and the representation 204 may be labeled Device 2 to show association with the second computing device 106. The representation 206 is arranged within the box of the representation 202 to reflect that both Programs A and B are operated by the first computing device 106. The configuration items 110 indicated by the representations 202, 204, 206 shown in FIG. 2 may be a tiny sub-set of the CMDB 200 for the entire pharmaceutical network 100. The CMDB 200 provides information to the operator, such that the first computing device 106 includes and/or operates both computer programs (e.g., Programs A and B). Although variables are used in FIG. 2, the CMDB 200 may include specific identifying information for the computing devices 106 and the computer programs. For example, the CMDB 200 may include, for each computing device 106, a unique identifier and/or name, a type of the device (e.g., make, model, year), a location of the device (e.g., IP address, states, etc.), an owner of the device, a role of the device, and/or the like.

The CMDB 200 in FIG. 2 has several information gaps. For example, the CMDB 200 does not indicate a relationship between the two computing devices 106. The CMDB 200 also fails to state which computer program(s) are operated by the second computing device 106. The monitoring device 102 of the network management system may perform the automated discovery process described herein to fill-in missing information in the CMDB 200, thereby augmenting the CMDB 200 to provide additional information.

FIG. 3 is a block diagram of a portion of an updated CMDB 300 at a second time after the monitoring device 102 performs the automated discovery process according to an embodiment. The updated CMDB 300 in FIG. 3 may be an updated version of the CMDB 200 in FIG. 2. Through the automated discovery process, the monitoring device 102 determines a first relationship between the first and second computing devices 106. The monitoring device 102 may store a representation 302 of the first relationship (“relationship X” in FIG. 3) in the CMDB 300. The monitoring device 102 determines a second relationship between the second computing device 106 and a second computer program (“Program B”). For example, the second relationship indicates that the second computing device 106 operates Program B. The monitoring device 102 may store a representation 304 of Program B in the CMDB 300. The first and second relationships may be determined by the monitoring device 102 based on analysis of human-readable text in obtained messages, as described above. The updated CMDB 300 includes more information than the pre-existing CMDB 200 in FIG. 2, such that the updated CMDB 300 may be a better tool to use for tracking and management of pharmaceutical network 100 operations. For example, if the second computing device 106 malfunctions and/or is replaced, the known relationship between the first and second computing devices 106 indicates whether or not, and how, the first computing device 106 would be impacted by the removal of the second computing device 106. The updated CMDB 300 may be displayed on a display device of the I/O device 128 or another one of the computing devices 106, to enable an operator to observe the configuration items 110 and the known relationships between the configuration items 110.

After updating the CMDB 300, the monitoring device 102 may generate a notification indicating that the CMDB 300 is updated. The monitoring device 102 may communicate the notification, via the communication device 130 thereof, to at least one of the computing devices 106 of the pharmaceutical network 100. For example, the monitoring device 102 may communicate the notification that the CMDB 300 is updated to one or more of the pharmacy devices 112, benefit manager devices 114, client devices 118, IT support devices 132, and/or personal devices 120. The monitoring device 102 optionally may generate a record of the CMDB 300 update and store the record in a remote server 122 and/or the local memory device 126 of the monitoring device 102.

In an embodiment, the monitoring device 102 may perform an inverted discovery process, relative to the discovery process described herein. For example, the monitoring device 102 may review the existing CMDB 104 to search for information gaps. With reference to FIG. 2, the monitoring device 102 may detect that a relationship between the first and second computing devices 106 is absent. The monitoring device 102 may also detect that the CMDB 200 fails to list any computer programs that are operated by the second computing device 106. After determining which relationships are absent from the CMDB 104, the monitoring device 102 may obtain messages associated with the configuration items 110 involved in the absent relationships. In this example, the monitoring device 102 may specifically target messages generated by and/or sent to the second computing device 106. The monitoring device 102 may detect and then analyze human-readable text in the messages obtained. The monitoring device 102 may attempt to ascertain, via the content (e.g., meaning and context) of the human-readable text, what the relationship is between the second computing device 106 and the first computing device 106, as well as which computer programs are operated by the second computing device 106. In this embodiment, the monitoring device 102 may first determine which relationships are absent or appear incorrect in the CMDB 104, and then may target messages to analyze to attempt to determine the missing information. Once the monitoring device 102 resolves an absent relationship, the monitoring device 102 may update the CMDB 104 to include a representation of the relationship that is determined (e.g., representations 302 and 304 shown in FIG. 3). The monitoring device 102 may periodically perform the inverted discovery process as well as periodically performing the automated discovery process described above. Optionally, the monitoring device 102 may perform the inverted discovery process on demand upon receipt of a command message.

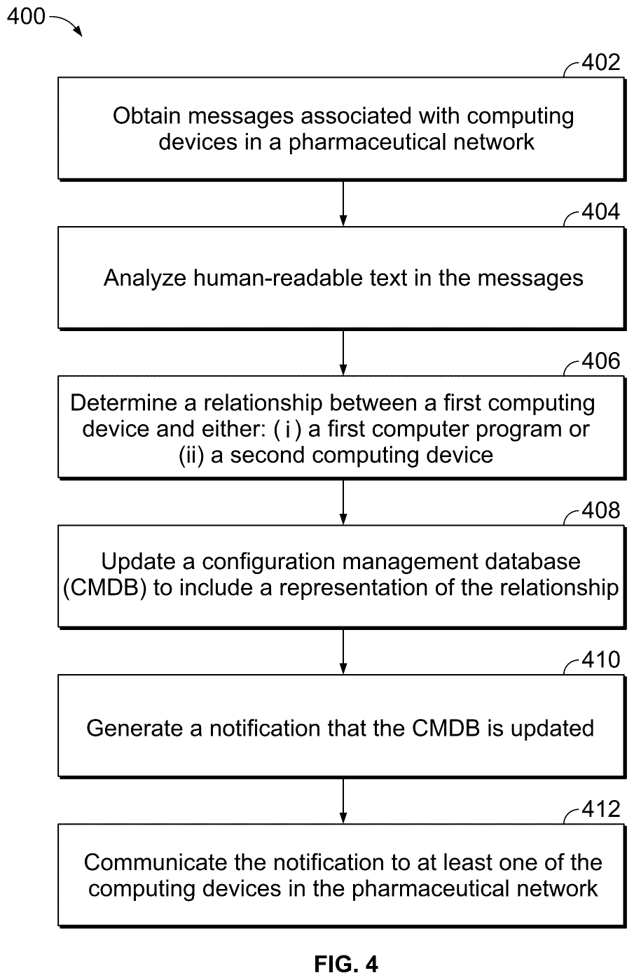

FIG. 4 illustrates a flowchart of one example of a method 400 for automated relationship discovery in a pharmaceutical network 100. The method can represent operations performed by the monitoring device 102 of the network management system shown in FIG. 1. At step 402, messages are obtained which are associated with computing devices 106 in a pharmaceutical network 100. For example, obtaining the messages may include obtaining prescription-related orders, communications generated and transmitted from the first computing device 106, communications generated and transmitted from the second computing device 106, engineer notes, and/or information technology (IT) service requests. The computing devices 106 to which the messages are associated may include at least two of: a pharmacy device 112, a benefit manager device 114, a fulfillment device 116, an IT support device 132, and a client device 118. The messages optionally may be obtained by accessing email messages within an email account that is associated with a user of a first computing device 106. Optionally, at least some of the messages that are obtained may be generated using an application, and the obtaining the messages may be accomplished by accessing the messages that are generated by a first computing device 106 using the application.

At step 404, human-readable text within the messages that are obtained are analyzed. For example, human-readable text of a first message may be within one or more of (i) a sender field of the first message, (ii) a recipient field of the first message, (iii) a subject field of the first message, (iv) a body field of the first message, or (v) a signature field of the first message. Optionally, metadata of the first message may be inspected for the presence of human-readable text. If present, the human-readable text of the metadata may be analyzed. The analysis of the human-readable text may be performed by implementing one or more natural language processing techniques. Additionally or alternatively, the analysis may include providing the human-readable text as an input to a machine learning algorithm.

At step 406, a relationship is determined between a first computing device 106 and either: (i) a first computer program operated by the first computing device 106, or (ii) a second computing device 106 of the computing devices 106. The relationship is determined based on the analysis of the human-readable text.

At step 408, a CMDB 104 is updated to include a representation of the relationship that is determined. The CMDB 104 may be updated by generating a new association between two configuration items 110 in the CMDB 104 and/or validating an existing association between the two configuration items 110 in the CMDB 104. The representation of the relationship may be depicted in a GUI that is displayed on a display device for observation by an operator. For example, the newly-added representation of the relationship may be highlighted in the GUI to draw attention to the updated information in the CMDB 104.

At step 410, a notification is generated that indicates that the CMDB 104 is updated. At step 412, the notification is communicated to at least one of the computing devices 106 of the pharmaceutical network 100.

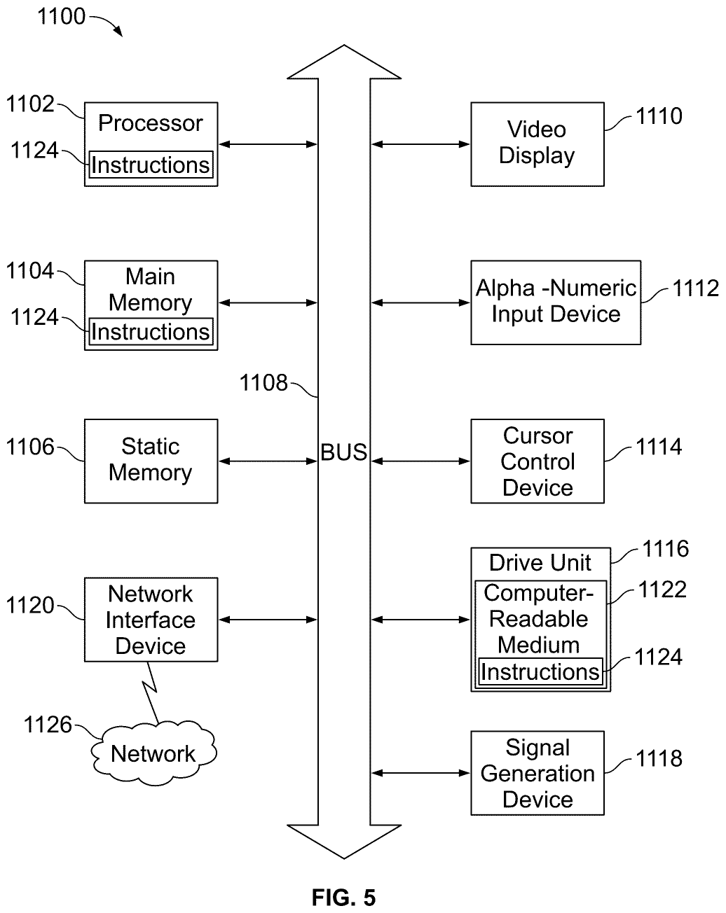

FIG. 5 shows a block diagram of the monitoring device 102 (shown in FIG. 1) in the example form of a computer system 1100 within which a set of instructions may be executed causing the machine to perform any one or more than one methods, processes, operations, or methodologies discussed herein. The computing devices 106, for example, may include the functionality of the one or more than one computer systems 1100. These devices and systems are dedicated to performing any one or more than one methods, processes, operations, or methodologies discussed herein.

In an example embodiment, the machine operates as a standalone device or may be connected (e.g., networked, etc.) to other machines. In a networked deployment, the machine may operate in the capacity of a server or a client machine in server-client network environment, or as a peer machine in a peer-to-peer (or distributed) network environment.

The example computer system 1100 includes one or more processors 1102 (e.g., a central processing unit (CPU), a graphics processing unit (GPU) or both, etc.), a main memory 1104 and a static memory 1106, which communicate with each other via a bus 1108. The computer system 1100 further includes a video display unit 1110 (e.g., a liquid crystal display (LCD) or a cathode ray tube (CRT), etc.). The computer system 1100 also includes an alphanumeric input device 1112 (e.g., a keyboard, etc.), a cursor control device 1114 (e.g., a mouse, etc.), a drive unit 1116, a signal generation device 1118 (e.g., a speaker, etc.) and a network interface device 1120.

The drive unit 1116 includes a computer readable medium 1122 on which is stored one or more than one sets of instructions 1124 (e.g., software, etc.) embodying any one or more than one methodologies or functions described herein. The instructions 1124 may also reside, completely or at least partially, within the main memory 1104 and/or within the processor 1102 during execution thereof by the computer system 1100, the main memory 1104 and the processor 1102 also constituting non-transitory computer readable media. When loaded with the instructions 1124, the processor 1102 is a machine dedicated to only the present processes and methodologies. The instructions 1124 may further be transmitted or received over a network 1126 via the network interface device 1120.

While the computer-readable medium 1122 is shown in an example embodiment to be a single medium, the term “computer-readable medium” should be taken to include a single medium or multiple media (e.g., a centralized or distributed database, and/or associated caches and servers, etc.) that store the one or more than one sets of instructions. The term “computer-readable medium” shall also be taken to include any medium that is capable of storing or encoding a set of instructions for execution by the machine and that cause the machine to perform any one or more than one methodologies of the present invention. The term “Computer-readable medium” shall accordingly be taken to include, but not be limited to, solid-state memories, optical media, and magnetic media. In some embodiments, the computer-readable medium is a non-transitory computer-readable medium. In other examples, a computer-readable medium is any medium that satisfies statutory requirements and stores instructions for use by a machine.

As described above, all or part of the systems described herein may be an artificial intelligence (AI) or machine-learning system that can automatically perform the operations of the methods also described herein. These types of systems may be trained from outside information and/or self-trained to repeatedly improve the accuracy with how human-readable text in messages is interpreted to discern relationships between computing devices and computer programs in the pharmaceutical network. Over time, these systems can improve by determining relationships, based on human-readably text in messages, with increasing accuracy and speed, thereby significantly enhancing the amount of information and accuracy of the information stored in the CMDB, which improves management of the pharmaceutical network operations. The AI or machine-learning systems described herein may include technologies enabled by adaptive predictive power and that exhibit at least some degree of autonomous learning to automate and/or enhance pattern detection (e.g., recognizing irregularities or regularities in the human-readable text within messages), customization (e.g., generating or modifying rules to optimize relationship discovery through text), or the like.

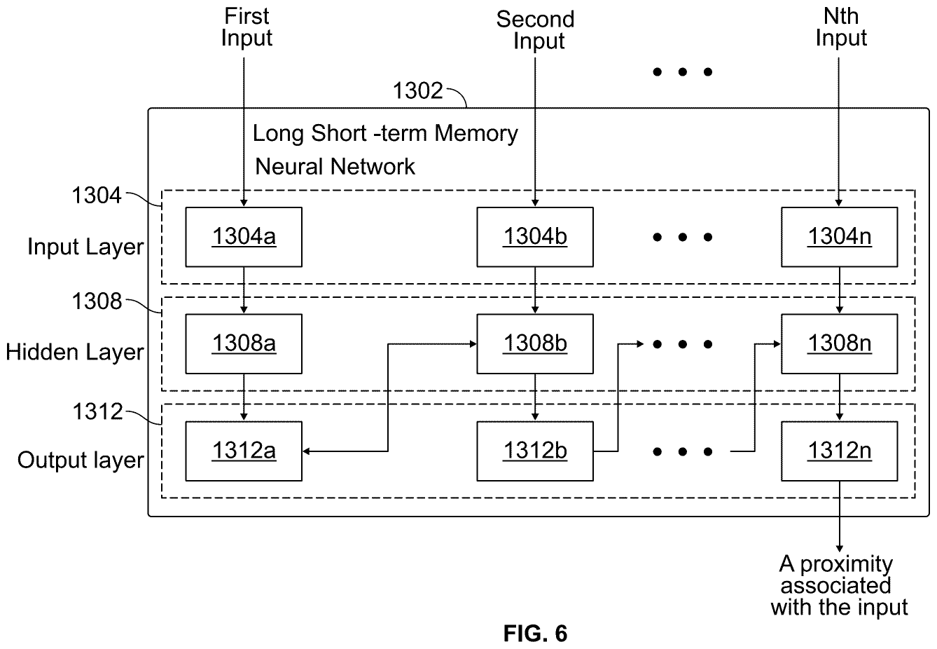

FIG. 6 illustrates a functional block diagram of an example neural network 1302 that can be used by the monitoring device 102 for analyzing human-readable text within messages to determine relationships among the computing devices 106 and computer programs in the pharmaceutical network 100. The monitoring device 102 may update the CMDB 104 to include information about the relationships that are discerned from the analysis, as described herein. In an example, the neural network 1302 can represent a long short-term memory (LSTM) neural network. In an example, the neural network 1302 can represent one or more recurrent neural networks (RNN). The neural network 1302 may be used to implement the machine learning algorithm as described herein, and various implementations may use other types of machine learning networks. The neural network 1302 includes an input layer 1304, one or more intermediate or hidden layers 1308, and an output layer 1312. Each layer 1304, 1308, 1312 includes individual units, or neurons. Each neuron can receive information (e.g., as input into the neural network 1302 or as received as output from another neuron in another layer or the same layer), process this information to generate output, and provide the output to another neuron or as output of the neural network 1302. The input layer 1304 may include several input neurons 1304a, 1304b, . . . 1304n, the hidden layer 1308 may include several intermediate neurons 1308a, 1308b, . . . 1308n, and the output layer 1312 may include several output neurons outputs 1312a, 1312b, . . . 1312n. The input may include, for example, human-readable text of the messages associated with one or more of the computing devices 106 in the pharmaceutical network 100.

Each neuron can receive an input from another neuron and output a value to another neuron (e.g., in the output layer 1312 or another layer). Each neuron may receive an output of a previous neuron as an input. For example, the intermediate neuron 1308a can receive an input from the input neuron 1304a and output a value to the output neuron 1312a. Similarly, the intermediate neuron 1308b may receive input from the input neuron 1304b and output a value to the output neuron 1312a. The outputs of the neurons may be fed forward to another neuron in the same or different intermediate layer 1308.

The processing performed by the neurons may vary based on the neuron but can include the application of the various rules or criteria described herein to partially or entirely decide whether the human-readable text in one or more messages demonstrates a relationship between a first computing device and at least one of a second computing device or a computer program operated by the first computing device. The output of the application of the rule or criteria can be passed to another neuron as input to that neuron. One or more neurons in the intermediate and/or output layers 1308, 1312 can determine that a relationship is indicated by the human-readable text or is not indicated. The last output neuron 1312n in the output layer 1312 may output a relationship or no-relationship decision. For example, the output from the neural network 1302 can be an indication of a relationship between two computing devices, or that a first computing device is operating a first computer program. Alternatively, the output can be a probability of a relationship. Although the input layer 1304, the intermediate layer(s) 1308, and the output layer 1312 are depicted as each including three artificial neurons, one or more of these layers may contain more or fewer artificial neurons. The neurons can include or apply one or more adjustable parameters, weights, rules, criteria, or the like, as described herein, to perform the processing by that neuron.

In some embodiments, the neural network 1302 may be a convolutional neural network. In a convolutional neural network, however, the output layer may include one fewer output neuron than the number of neurons in the intermediate layer(s), and each neuron may be connected to each output neuron. Additionally, each input neuron in the input layer may be connected to each neuron in the hidden or intermediate layer(s).

The neural network 1302 or other machine-learning algorithm may be trained and re-trained using feedback from one or more prior analyses of the human-readable text within one or more messages. This feedback may indicate missed relationships or incorrectly identified relationships between computing devices and/or computer programs operated by the computing devices. Based on this feedback, the network management system 123 (e.g., including the neural network 1302 or other machine learning algorithm) may be trained by adjusting one or more parameters, weights, rules, criteria, or the like, used in the analysis of the same or other messages to better identify established relationships (e.g., by reducing missed and incorrect relationships). This process can be performed using production data instead of training data and may be repeated many times to repeatedly improve the determination of relationships based on human-readable text in messages. The training of the AI or machine-learning system may minimize false positives (e.g., over-detection of relationships) and/or false negatives (e.g., under-detection of relationships) by performing an iterative training algorithm, in which the AI or machine-learning system is retrained with an updated set of production data and based on the feedback from the messages analyzed prior to the most recent training of the AI or machine-learning system. This provides a robust relationship discovery model.

The network management systems and methods described herein provide a technical improvement over conventional database maintenance techniques by providing a new resource for determining relationships between configuration items in the CMDB. The new resource involves automated analysis of human-readable text within messages associated with computing devices in the network. The conventional database techniques do not utilize this resource to determine information for updating a database. Using this new resource to extract information solves a persistent problem in the management of CMDB for large networks, which is determining the appropriate information to fill in information gaps in the CMDB to provide a more robust and up-to-date CMDB. The network management system and method may be able to ascertain relationships, from the analysis of the messages, that would not be achievable using conventional resources for updating a CMDB. As such, the embodiments described herein do not merely represent an improvement in kind over conventional database management techniques, but rather an entire shift or step change in how the information for updating the CMDB is determined. The network management systems and methods represent an advancement in computer technology by automatically updating a CMDB based on relationships learned via human-readable text within low level information communicated within a network.

Furthermore, the network management systems and methods provide a technical improvement for significantly increasing the efficiency, accuracy, and timeliness of database updates, relative to conventional manual entry. Compared to prior art and conventional database updating techniques, the subject matter described herein determines relationships between configuration items and updates the CMDB to include representations of the relationships in less time, with less user interaction, and with fewer errors.

In one or more embodiments, the CMDB is not a generic, standard database. For example, the CMDB may be a particular type of database that describes the specific components of a pharmaceutical network and relationships between the components. The network management systems and methods may be specifically directed to managing and updating the CMDB.

One or more embodiments of the subject matter described herein involve a computer's utilization of a machine learning algorithm or model to analyze many messages received from multiple, different sources to determine, based on human-readable text within the messages, how computer devices within a pharmaceutical network are related to each other and to computer programs operated by the computer devices. While some examination of messages can be performed mentally or with pen and paper, the subject matter described herein may transform (through training or machine learning) a neural network and data contained in the messages to extract information about the relationships between computing devices and/or computer programs that is beyond the capability of a human operator. For example, due to the greater processing speed and memory storage capacity of the network management system over the human operator, the network management system may be able to recognize associations from text in the messages that a human operator would fail to perceive.

In the foregoing Detailed Description, it can be seen that various features are grouped together in a single embodiment for the purpose of streamlining the disclosure. This method of disclosure is not to be interpreted as reflecting an intention that the claimed embodiments require more features than are expressly recited in each claim. Rather, as the following claims reflect, present disclosure may lie in less than all features of a single disclosed embodiment. Thus, the following claims are hereby incorporated into the Detailed Description, with each claim standing on its own as a separate embodiment.

The foregoing description is merely illustrative in nature and is in no way intended to limit the disclosure, its application, or uses. The broad teachings of the disclosure can be implemented in a variety of forms. Therefore, while this disclosure includes particular examples, the true scope of the disclosure should not be so limited since other modifications will become apparent upon a study of the drawings, the specification, and the following claims. It should be understood that one or more steps within a method may be executed in different order (or concurrently) without altering the principles of the present disclosure. Further, although each of the embodiments is described above as having certain features, any one or more of those features described with respect to any embodiment of the disclosure can be implemented in and/or combined with features of any of the other embodiments, even if that combination is not explicitly described. In other words, the described embodiments are not mutually exclusive, and permutations of one or more embodiments with one another remain within the scope of this disclosure.

Spatial and functional relationships between elements (for example, between modules) are described using various terms, including “connected,” “engaged,” “interfaced,” and “coupled.” Unless explicitly described as being “direct,” when a relationship between first and second elements is described in the above disclosure, that relationship encompasses a direct relationship where no other intervening elements are present between the first and second elements, and also an indirect relationship where one or more intervening elements are present (either spatially or functionally) between the first and second elements. As used herein, the phrase at least one of A, B, and C should be construed to mean a logical (A OR B OR C), using a non-exclusive logical OR, and should not be construed to mean “at least one of A, at least one of B, and at least one of C.”

The term “based on” or using, as used herein, reflects an open-ended term that can reflect others elements beyond those explicitly recited.

In the figures, the direction of an arrow, as indicated by the arrowhead, generally demonstrates the flow of information (such as data or instructions) that is of interest to the illustration. For example, when element A and element B exchange a variety of information but information transmitted from element A to element B is relevant to the illustration, the arrow may point from element A to element B. This unidirectional arrow does not imply that no other information is transmitted from element B to element A. Further, for information sent from element A to element B, element B may send requests for, or receipt acknowledgements of, the information to element A. The term subset does not necessarily require a proper subset. In other words, a first subset of a first set may be coextensive with (equal to) the first set.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

The computer programs include processor-executable instructions that are stored on at least one non-transitory computer-readable medium. The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc.

The computer programs may include: (i) descriptive text to be parsed, such as HTML (hypertext markup language), XML (extensible markup language), or JSON (JavaScript Object Notation), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective C, Swift, Haskell, Go, SQL, R, Lisp, Java®, Fortran, Perl, Pascal, Curl, OCaml, Javascript®, HTML5 (Hypertext Markup Language 5th revision), Ada, ASP (Active Server Pages), PUP (PUP: Hypertext Preprocessor), Scala, Eiffel, Smalltalk, Erlang, Ruby, Flash®, Visual Basic®, Lua, MATLAB, SIMULINK, and Python®.

None of the elements recited in the claims is intended to be a means-plus-function element within the meaning of 35 U.S.C. § 112(f) unless an element is expressly recited using the phrase “means for,” or in the case of a method claim using the phrases “operation for” or “step for.”

The methods described herein do not have to be executed in the order described, or in any particular order. Moreover, various activities described with respect to the methods identified herein can be executed in serial or parallel fashion. Although “End” blocks may be shown in the flowcharts, the methods may be performed continuously.

In the foregoing Detailed Description, it can be seen that various features are grouped together in a single embodiment for the purpose of streamlining the disclosure. This method of disclosure is not to be interpreted as reflecting an intention that the claimed embodiments require more features than are expressly recited in each claim. Rather, as the following claims reflect, present disclosure may lie in less than all features of a single disclosed embodiment. Thus, the following claims are hereby incorporated into the Detailed Description, with each claim standing on its own as a separate embodiment.

What is claimed is:

1. A method for automated discovery of component relationships in a pharmaceutical network, the method comprising:
obtaining messages associated with computing devices in a pharmaceutical network;
analyzing, via a monitoring device comprising one or more processors, human-readable text in the messages;
determining, via the monitoring device, a relationship between a first computing device of the computing devices and one of (i) a first computer program operated by the first computing device or (ii) a second computing device of the computing devices, based on the analysis of the human-readable text; and
updating a configuration management database (CMDB) to include a representation of the relationship that is determined.

2. The method of claim 1, wherein obtaining the messages comprises obtaining one or more of prescription-related orders, communications generated and transmitted from the first computing device, communications generated and transmitted from the second computing device, engineer notes, or information technology (IT) service requests.

3. The method of claim 1, wherein obtaining the messages associated with the computing devices in the pharmaceutical network comprises obtaining the messages associated with at least two of: a pharmacy device, a benefit manager device, a fulfillment device, an IT support device, or a client device.

4. The method of claim 1, wherein analyzing the human-readable text in the messages comprises implementing one or more natural language processing techniques.

5. The method of claim 1, wherein analyzing the human-readable text in the messages comprises providing the human-readable text as an input to a machine learning algorithm.

6. The method of claim 1, wherein analyzing the human-readable text in the messages comprises analyzing human-readable text within one or more of: (i) a sender field of a first message of the messages, (ii) a recipient field of the first message, (iii) a subject field of the first message, (iv) a body field of the first message, or (v) a signature field of the first message.

7. The method of claim 1, wherein analyzing the human-readable text in the messages comprises analyzing human-readable text within metadata of a first message of the messages.

8. The method of claim 1, further comprising:
generating a notification indicating that the CMDB is updated; and
communicating the notification to at least one of the computing devices of the pharmaceutical network.

9. The method of claim 1, wherein obtaining the messages comprises accessing email messages within an email account that is associated with a user of the first computing device.

10. The method of claim 1, wherein the first computer program operated by the first computing device is an application, and obtaining the messages comprises accessing messages generated by the first computing device using the application.

11. The method of claim 1, wherein updating the CMDB to include the representation of the relationship comprises one of (i) generating a new association between two configuration items in the CMDB or (ii) validating an existing association between the two configuration items in the CMDB.

12. A network management system for a pharmaceutical network, the network management system comprising:
a configuration management database (CMDB) that contains information related to computing devices in a pharmaceutical network, computer programs operated by the computing devices, and relationships among the computing devices and the computer programs; and
a monitoring device comprising one or more processors and communicatively connected to the CMDB, the monitoring device configured to obtain messages associated with the computing devices in the pharmaceutical network and analyze human-readable text in the messages, the monitoring device configured to determine a relationship between a first computing device of the computing devices and one of (i) a first computer program operated by the first computing device or (ii) a second computing device of the computing devices, based on the analysis of the human-readable text, the monitoring device configured to update the CMDB to include a representation of the relationship that is determined.

13. The network management system of claim 12, wherein the computing devices with which the messages are associated include at least two of: a pharmacy device, a benefit manager device, a fulfillment device, an IT support device, or a client device.

14. The network management system of claim 12, wherein the messages that are obtained comprise one or more of prescription-related orders, communications generated and transmitted from the first computing device, communications generated and transmitted from the second computing device, engineer notes, or information technology (IT) service requests.

15. The network management system of claim 12, wherein the monitoring device is configured to analyze the human-readable text in the messages by at least one of: (i) implementing one or more natural language processing techniques, or (ii) providing the human-readable text as an input to a machine learning algorithm.

16. The network management system of claim 12, wherein the monitoring device is configured to analyze at least some of the human-readable text within one or more of: (i) a sender field of a first message of the messages, (ii) a recipient field of the first message, (iii) a subject field of the first message, (iv) a body field of the first message, (v) a signature field of the first message, or (vi) metadata of the first message.

17. The network management system of claim 12, wherein the monitoring device is configured to generate a notification indicating that the CMDB is updated, and communicate the notification to at least one of the computing devices of the pharmaceutical network.

18. The network management system of claim 12, wherein the first computer program operated by the first computing device is an application, and the monitoring device is configured to obtain the messages by accessing messages generated by the first computing device using the application.

19. The network management system of claim 12, wherein the monitoring device is configured to update the CMDB to include the representation of the relationship by one of (i) generating a new association between two configuration items in the CMDB or (ii) validating an existing association between the two configuration items in the CMDB.

20. A computer program product comprising a non-transitory computer readable storage medium, the non-transitory computer readable storage medium comprising computer executable code configured to be executed by one or more processors to:
obtain messages associated with computing devices in a pharmaceutical network;
analyze human-readable text in the messages;
determine a relationship between a first computing device of the computing devices and one of (i) a first computer program operated by the first computing device or (ii) a second computing device of the computing devices, based on the analysis of the human-readable text; and
update a configuration management database (CMDB) to include a representation of the relationship that is determined.
